(19) United States
(12) Patent Application Publication
(10) Pub. No.: US 2005/0165073 A1
(43) Pub. Date: Jul. 28, 2005

(54) Isothiazolone composition and method for stabilizing isothiazolone

(76) Inventors: Wan-Pyo Hong, Gyeonggi-do (KR); Jin-Man Kim, Gyeonggi-do (KR); Ki-Seung Choi, Gyeonggi-do (KR); Jin-Ho Kim, Gyeonggi-do (KR); Ki-Man Park, Gyeonggi-do (KR); Hi-Weon Jung, Gyeonggi-do (KR)

Correspondence Address: GIFFORD, KRASS, GROH, SPRINKLE & CITKOWSKI, P.C, PO BOX 7021, TROY, MI, 48007-7021, US

(21) Appl. No.: 10/515,851

(22) PCT Filed: May 28, 2003
(86) PCT No.: PCT/KR03/01047

(30) Foreign Application Priority Data
May 29, 2002 (KR) 1020020030023
Oct. 11, 2002 (KR) 1020020062096

(51) Int. Cl. A61K 31/425

(52) U.S. Cl. 514/372

(57) ABSTRACT

The present invention relates to an isothiazolone composition and a method for stabilizing the same, and particularly to an isothiazolone composition that does not comprise alkali earth metal salt of a divalent metal such as magnesium chloride, which causes salt shock, and thus that can be stably used as a sterilizer in a dispersion or emulsion such as paint acrylate or latex, therefore effectively preventing growth of microorganisms, and that has superior storage stability due to its low precipitation formation rate because it comprises a precipitation-preventing agent, and a method for stabilizing the same.

### BACKGROUND OF THE INVENTION

(a) Field of the Invention

The present invention relates to an isothiazolone composition and a method for stabilizing the same, and more specifically to an isothiazolone composition that does not comprise a divalent alkali earth metal salt such as magnesium chloride, which causes salt shock, and thus can be stably used for a sterilizer in a dispersion or an emulsion such as paint acrylate or latex, etc., therefore effectively preventing the growth of microorganisms, and that has superior storage stability due to its low rate of precipitation formation.

(b) Description of the Related Art

Isothiazolone, which has extensive sterilizing activity for many harmful organisms originating from animals and plants such as bacteria, mold, algae, etc., is added to cosmetics, paints, etc. as an inhibitor for the growth of harmful microorganisms.

However, since isothiazolone is decomposed in a polar organic solvent such as alcohol, or in water, its biological activity deteriorates with the passage of time. Therefore, many studies for increasing stability of isothiazolone in a polar organic solvent or water have been conducted.

In order to increase stability of isothiazolone in a polar organic solvent or water, U.S. Pat. No. 3,870,785 has suggested a method of adding a metal nitrate salt, a nitrite salt, etc. to the composition as a stabilizer. And, U.S. Pat. No. 4,165,318 has suggested a method of adding formaldehyde or a formaldehyde-eluting material to an isothiazolone composition as a stabilizer. In addition, U.S. Pat. No. 4,824,957 has suggested a method of adding an organic hydroxylic solvent to an isothiazolone composition as a stabilizer.

However, for various reasons, it is preferable to reduce the amount of the stabilizer used for stabilizing a sterilizer comprising an isothiazolone compound.

For example, a dispersion or an emulsion such as paint acrylate or latex, etc., which requires prevention of change and decomposition by microorganisms, sensitively reacts with a salt, and particularly, it does not mix well with a sterilizer comprising salts of divalent ions which cause coagulation. Moreover, sterilization active ingredients may be incorporated into the coagulation, thus the sterilization active ingredient may be reduced or removed if a product to be stored is filtered.

At present, a metal nitrate salt, particularly magnesium chloride or magnesium nitrate, is added to a 3-isothiazolone composition as a stabilizer. A commonly used isothiazolone composition comprises 1 to 20 wt % of isothiazolone and 15 to 25 wt % of a nitrate salt.

In addition, isothiazolone compositions comprise divalent metal, particularly magnesium oxide, as a pH-neutralizing agent. However, an isothiazolone composition comprising a substantial amount of a divalent metal salt such as magnesium oxide cannot be preferably used for a sterilizer because it has a problem due to “salt shock” of bivalent metal salts.

### SUMMARY OF THE INVENTION

The present invention relates to an isothiazolone composition and a method for stabilizing the same, and more specifically to an isothiazolone composition that does not include a divalent alkali earth metal salt such as magnesium chloride, which causes salt shock, and thus that can be stably used for a sterilizer in a dispersion or an emulsion such as paint acrylate or latex, etc., therefore effectively preventing the growth of microorganisms, and that has superior storage stability due to its low rate of precipitation formation.

### BRIEF DESCRIPTION OF THE DRAWINGS

FIG. 1 is a photograph showing salt shock results of isothiazolone compositions of Example 1, Comparative Example 1, and a Control according to Experiment 1.

FIG. 2 is a photograph showing salt shock results of 10-fold dilutions of the isothiazolone compositions of Example 1 and Comparative Example 1 according to Experiment 1.

FIG. 3 is a photograph showing salt shock results of isothiazolone compositions of Example 1 and Comparative Example 1 according to Experiment 2 (paint acrylate emulsion).

FIG. 4 is a photograph showing the salt shock results of the isothiazolone compositions of Example 1 and Comparative Example 1 according to Experiment 3 (latex emulsion).

### DETAILED DESCRIPTION OF THE PREFERRED EMBODIMENTS

The present invention provides a stable isothiazolone composition that does not include an alkali earth metal salt of a divalent metal such as magnesium chloride, which causes salt shock, and thus can be stably used for a microorganism inhibitor in a dispersion or an emulsion such as paint acrylate or latex, etc., therefore effectively preventing the growth of harmful microorganisms, and that has superior storage stability because it includes appropriate ratios of three kinds of stabilizers having synergy for increasing stability of an isothiazolone solution, and thus has a low rate of precipitation formation even at a much lower concentration than those disclosed in the prior art.

A method for stabilizing isothiazolone is also disclosed.

The present invention provides an isothiazolone composition including (a) a 3-isothiazolone compound represented by the following Chemical Formula 1; (b) an alkali metal nitrate salt; (c) an alkali metal chloride salt; and (d) a solvent:

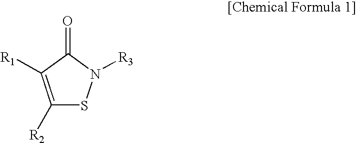

In the above Chemical Formula 1,
R₁ and R₂ are each independently a hydrogen atom, a halogen atom, or a C₁-C₄ alkyl group or an aryl group; and,
R₃ is a hydrogen atom, a C₁-C₁₈ alkyl group, a C₂-C₁₈ alkenyl group, a C₂-C₁₈ alkynyl group, a C₃-C₁₂ cycloalkyl group having trigonal to octagonal rings, a C₁₀-C₂₄ aralkyl group, or a C₁₀-C₂₄ aryl group.

The present invention also provides a method for stabilizing isothiazolone comprising adding (a) 0.1 to 20 wt % of a 3-isothiazolone compound represented by the above Chemical Formula 1; (b) 0.1 to 25 wt % of an alkali metal nitrate salt; and (c) 0.001 to 20 wt % of an alkali metal carbonate salt to (d) a solvent making up the balance to prepare a mixed solution:

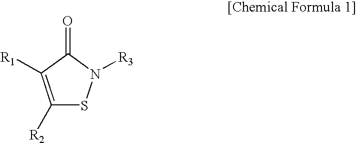

In the above Chemical Formula 1,
R₁ and R₂ are independently a hydrogen atom, a halogen atom, or a C₁-C₄ alkyl group or aryl group; and,
R₃ is a hydrogen atom, a C₁-C₁₈ alkyl group, a C₂-C₁₈ alkenyl group, a C₂-C₁₈ alkynyl group, a C₃-C₁₂ cycloalkyl group having trigonal to octagonal rings, a C₁₀-C₂₄ aralkyl group, or a C₁₀-C₂₄ aryl group.

The present invention will now be explained in detail.

The present invention is characterized by including (a) a 3-isothiazolone compound represented by the following Chemical Formula 1; (b) an alkali metal nitrate salt; (c) an alkali metal chloride salt; and (d) a solvent as essential ingredients:

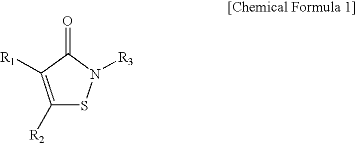

In the above Chemical Formula 1,
R₁ and R₂ are independently a hydrogen atom, a halogen atom, a C₁-C₄ alkyl group or aryl group;
R₃ is a hydrogen atom, a C₁-C₁₈ alkyl group, a C₂-C₁₈ alkenyl group, a C₂-C₁₈ alkynyl group, a C₃-C₁₂ cycloalkyl group having trigonal to octagonal rings, a C₁₀-C₂₄ aralkyl group, or C₁₀-C₂₄ aryl group.

The (a) 3-isothiazolone compound, which has extensive sterilizing activity for many harmful microorganisms originating from animals and plants such as bacteria, mold, algae, etc., is added to cosmetics, paints, etc. to be used for an inhibitor for growth of harmful microorganisms.

The (a) 3-isothiazolone compound of the above Chemical Formula 1 is preferably used in an amount of 0.1 to 20 wt % in the isothiazolone composition of the present invention. If the content of the (a) 3-isothiazolone compound is less than 0.1 wt %, the resulting isothiazolone composition cannot be commercially used because of a low content of sterilizer. And, if the content exceeds 20 wt %, stability deteriorates.

As the (a) 3-isothiazolone compound, 5-chloro-2-methyl-4-isothiazolin-3-one, 2-methyl-4-isothiazolin-3-one, 4,5-dichloro-2-methyl-4-isothiazolin-3-one, 5-chloro-n-octyl-3-isothiazolone, 4,5-dichloro-2-n-octyl-3-isothiazolone, benzisothiazolone or a mixture thereof can be preferably used.

In the case where the (a) 3-isothiazolone compound of Chemical Formula 1 used is a mixture, 5-chloro-2-methyl-4-isothiazolonin-3-one and 2-methyl-4-isothiazolin-3-one is preferable, and the mixing ratio thereof is preferably 1:20 to 20:1.

In addition, the isothiazolone composition of the present invention includes the (b) alkali metal nitrate salt as a stabilizer for preventing decomposition of isothiazolone.

In order to prevent formation of a precipitation of the isothiazolone composition, an alkali earth metal salt of a divalent metal nitrate salt has been widely used, generally magnesium nitrate. However, if the content of the magnesium nitrate is less than 0.1 wt % of the isothiazolone composition, precipitation-preventing effects cannot be anticipated, and if it exceeds 25 wt %, solubility of magnesium nitrate decreases to cause precipitation of magnesium nitrate itself, and worse, divalent ion magnesium causes salt shock and prevents stabilization of aqueous polymer dispersions.

However, since the isothiazolone composition of the present invention includes an alkali metal nitrate salt as a stabilizer, instead of an alkali earth metal salt of a divalent metal such as magnesium nitrate that has been predominantly used as a stabilizer in an isothiazolone composition of the prior art, salt shock is inhibited which makes the isothiazolone composition stable.

The content of the (b) alkali metal nitrate salt is preferably 0.1 to 25 wt % of the isothiazolone composition. If the content is less than 0.1 wt %, precipitation of isothiazolone cannot be effectively prevented, and if it exceeds 25 wt %, solubility decreases and product cost increases, making the composition uneconomical.

As the (b) alkali metal nitrate salt, lithium nitrate, sodium nitrate, potassium nitrate, or a mixture thereof can be preferably used. Sodium nitrate is most preferable.

In addition, the isothiazolone composition of the present invention includes (c) an alkali metal chloride salt that is produced by the reaction of an alkali metal introduced as a pH neutralizing agent with chloric acid.

Magnesium oxide, which has been widely used as a pH neutralizing agent of an isothiazolone composition, reacts in the composition to form MgCl₂ (magnesium chloride). If the MgCl₂ exceeds 9 wt % of the composition, solubility of magnesium chloride decreases to cause precipitation of magnesium chloride itself, and more seriously, divalent metal magnesium causes salt shock and prevents stabilization of the aqueous polymer dispersion.

However, since the isothiazolone composition of the present invention includes an alkali metal carbonate salt as a pH-neutralizing agent, instead of magnesium oxide that has been predominantly used as a pH-neutralizing agent of an isothiazolone composition, salt shock is prevented.

The (c) alkali metal chloride salt is preferably present in an amount of 0.001 to 20 wt % of the isothiazolone composition, more preferably in an amount of 0.01 to 15 wt %, and most preferably 0.1 to 10 wt %. If the content is less than 0.001 wt %, the pH of the resulting isothiazolone composition cannot be controlled within a commercially acceptable range because the concentration of the alkali metal carbonate salt introduced as a pH-neutralizing agent is too low, and if the content exceeds 20 wt %, stability of the composition decreases and product cost increases, thus making the composition uneconomical.

The (c) alkali metal chloride salt is preferably sodium chloride, or potassium chloride with an alkali metal carbonate salt introduced thereto, or a mixture thereof. The alkali metal carbonate salt introduced as a pH-neutralizing agent is preferably sodium carbonate, potassium carbonate, sodium bicarbonate, potassium bicarbonate, or a mixture thereof.

In addition, the isothiazolone composition includes a (d) solvent. As the solvent, water, ethyleneglycol, propyleneglycol, dipropyleneglycol, polypropyleneglycol, diethyleneglycol, triethyleneglycol, 1,5-pentanediol, 2,4-pentanediol, benzyl alcohol or a mixture thereof can be preferably used.

As another preferred embodiment of the present invention, the isothiazolone composition of the present invention optionally further includes one of: (e) iodic acid, periodic acid, iodate, periodate or a mixture thereof; (f) a metal bromic acid; and (g) chlorite salt, alone or in combination of appropriate ratios, as a stabilizer for preventing the decomposition of isothiazolone. More preferably, a combination of the three kinds of precipitation-preventing agents (e)-(g) is added.

In addition, during the storage of the isothiazolone composition, a small amount of precipitation, which is different from that of salt shock, forms due to the decomposition of by-products during the production process and compounds produced during storage. The precipitation is commercially unfavorable because it damages the appearance of the product.

However, the present invention can effectively inhibit the precipitation by introducing a small amount of a mixture of three kinds of additives having superior effects for inhibiting formation of a precipitation to the isothiazolone composition. Specifically, the isothiazolone composition can be more effectively stabilized by using a mixture of compounds selected from a group consisting of (e) iodic acid, periodic acid, iodate salt, periodate salt, or a mixture thereof; (f) a metal bromic acid salt; and (g) chlorite salt, than by using the compound of (e), (f), and (g) alone.

The (e) iodic acid, periodic acid, iodate, periodate, or a mixture thereof is preferably present in an amount of 0.0001 to 0.01 wt % of the isothiazolone composition, more preferably 0.001 to 0.005 wt %. If the content is less than 0.0001 wt %, precipitation of the isothiazolone compound cannot be effectively prevented, and if it exceeds 0.01 wt %, production cost increases, thus making the composition uneconomical.

The (e) iodic acid, periodic acid, iodate, periodate, or a mixture thereof is preferably selected from a group consisting of lithium iodate, sodium iodate, potassium iodate, ammonium iodate, lithium periodate, sodium periodate, potassium periodate, ammonium periodate, iodic acid, periodic acid dihydrate, or a mixture thereof, and more preferably iodic acid, periodic acid dihydrate, sodium iodic acid, potassium iodic acid or a mixture thereof.

The (f) metal bromic acid, the other precipitation-preventing agent, is preferably present in an amount of 0.001 to 1.0 wt % of the isothiazolone composition, more preferably 0.01 to 0.2 wt %. If the content is less than 0.001 wt %, precipitation of isothiazolone cannot be effectively prevented, and if it exceeds 1.0 wt %, production cost increases, thus making the composition uneconomical.

The (f) metal bromic acid salt is preferably selected from a group consisting of sodium bromate, potassium bromate and a mixture thereof.

In addition, the (g) chlorite salt, the other precipitation-preventing agent, is preferably present in an amount of 0.001 to 10 wt %, more preferably 0.01 to 1.0 wt %, and most preferably 0.05 to 0.2 wt %. If the content is less than 0.001 wt %, precipitation of the isothiazolone cannot be effectively prevented, and if it exceeds 10 wt %, the production cost increases, thus making the composition uneconomical.

The (g) chlorite salt is preferably sodium chlorite, potassium chlorite or a mixture thereof.

The isothiazolone composition of the present invention preferably includes (a) 0. to 20 wt % of a 3-isothiazolone compound; (b) 0.1 to 25 wt % of an alkali metal nitrate salt; (c) 0.001 to 20 wt % of an alkali metal chloride salt; and (d) a solvent making up the balance, and the three kinds of precipitation-preventing agents of (e), (f), and (g) alone or in combination. The three kinds of precipitation-preventing agents preferably include (e) 0.0001 to 0.01 wt % of iodic acid, periodic acid, iodate, periodate, or a mixture thereof; (f) 0.01 to 1.0 wt % of a metal bromic acid salt; and (g) 0.001 to 10 wt % of chlorite salt.

The present invention also provides a method for stabilizing the isothiazolone composition comprising (i) adding (a) 0.1 to 20 wt % of a 3-isothiazolone compound represented by the following Chemical Formula 1; (b) 0.1 to 25 wt % of an alkali metal nitrate salt; and (c) 0.001 to 20 wt % of an alkali metal carbonate salt to (d) a solvent making up the balance, to prepare a mixed solution of the isothiazolone composition:

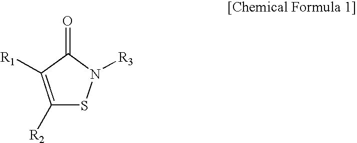

In the above Chemical Formula 1,
R₁ and R₂ are each independently a hydrogen atom, a halogen atom, or a C₁-C₄ alkyl group or aryl group; and
R₃ is a hydrogen atom, a C₁-C₁₈ alkyl group, a C₂-C₁₈ alkenyl group, a C₂-C₁₈ alkynyl group, a C₃-C₁₂ cycloalkyl group having trigonal to octagonal rings, a C₁₀-C₂₄ aralkyl group, or a C₁₀-C₂₄ aryl group.

The (a) 3-isothiazolone compound is preferably selected from a group consisting of 5-chloro-2-methyl-4-isothiazolin-3-one, 2-methyl-4-isothiazolin-3-one, 4,5-dichloro-2-methyl-4-isothiazolin-3-one, 5-chloro-2-n-octyl-3-isothiazolone, 4,5-dichloro-2-n-octyl-3-isothiazolone, benzisothiazolone, or a mixture thereof.

According to the method for stabilizing isothiazolone of the present invention, the (c) alkali metal carbonate salt is added as a pH-neutralizing agent. The introduced alkali metal carbonate salt reacts with chloric acid in the isothiazolone composition to exist in the form of an alkali metal chloride salt.

The (c) alkali metal carbonate is preferably introduced in an amount of 0.01 to 15 wt % of the isothiazolone composition, more preferably 0.1 to 10 wt %.

The (c) alkali metal carbonate is preferably sodium carbonate, potassium carbonate, sodium bicarbonate, potassium bicarbonate or a mixture thereof.

In addition, the (d) solvent is preferably selected from a group consisting of water, ethyleneglycol, propyleneglycol, dipropyleneglycol, polypropyleneglycol, diethyleneglycol, triethyleneglycol, 1,5-pentanediol, 2,4-pentanediol, benzyl alcohol or a mixture thereof.

As another preferred embodiment of the present invention, the method for stabilizing isothiazolone further adds a compound selected from a group consisting of (e) iodic acid, periodic acid, iodate, periodate or a mixture thereof; (f) a metal bromate; (g) chlorite salt, and a mixture thereof to the isothiazolone composition as a precipitation-preventing agent to further stabilize the isothiazolone composition. The mixture of two or more kinds of precipitation-preventing agents, which have different operation mechanisms, are preferably added in appropriate ratios.

The (e) iodic acid, periodic acid, iodate, periodate, or a mixture thereof is preferably added in an amount of 0.0001 to 0.01 wt % of the isothiazolone composition, more preferably 0.001 to 0.005 wt %. If the content is less than 0.0001 wt %, precipitation of isothiazolone cannot be effectively prevented, and if it exceeds 0.01 wt %, the color changes with the passage of time, and production cost increases thus making the method uneconomical. The (e) iodic acid, periodic acid, iodate, periodate or a mixture thereof is preferably selected from a group consisting of lithium iodate, sodium iodate, potassium iodate, ammonium iodate, lithium periodate, sodium periodate, potassium periodate, ammonium periodate, iodic acid, periodic acid dihydrate or admixture thereof, and iodic acid, periodic acid dihydrate, sodium iodic acid, potassium iodic acid or a mixture thereof are more preferable.

The (f) metal bromate is preferably added in an amount of 0.001 to 1.0 wt % of the isothiazolone composition, more preferably 0.001 to 0.2 wt %. If the content is less than 0.001 wt %, precipitation of isothiazolone cannot be effectively prevented, and if it exceeds 0.2 wt %, production cost increases, thus making the method uneconomical. The (f) metal bromate is preferably selected from a group consisting of sodium bromate, potassium bromate and a mixture thereof.

The (g) chlorite salt is preferably added in an amount of 0.001 to 10 wt % of the isothiazolone composition, more preferably 0.01 to 1.0 wt %, and most preferably 0.05 to 0.2 wt %. If the content is less than 0.001 wt %, precipitation of isothiazolone cannot be effectively prevented, and if it exceeds 10 wt %, production cost increases, thus making the method uneconomical. The (g) chlorite salt is preferably selected from sodium chlorite, potassium chlorite and a mixture thereof.

As explained, the isothiazolone composition of the present invention can be stably used in a dispersion or emulsion such as paint acrylate or latex, etc. because it does not include a divalent metal alkali earth metal salt such as magnesium chloride, which causes salt shock, therefore effectively inhibiting the growth of harmful microorganisms.

Accordingly, the isothiazolone composition of the present invention can be used for a microorganism inhibitor in microorganism habitats such as a cooling water tower, an air washer, a boiler, a mineral slurry, a wastewater disposal plant, a decorative fountain, a reverse osmosis filtration system, an ultrafiltration system, ballast water, an evaporation condenser, a heat exchanger, and for pulp and paper processing oils, plastics, emulsions and dispersants, paint, latex, coating agents, metal processing oils, etc., to effectively inhibit the growth of harmful microorganisms such as bacteria, mold, algae, etc.

The present invention will be explained with reference to the following Examples and Comparative Examples. However, these are intended to only illustrate the present invention and the present invention is not limited to them.

### EXAMPLES

Concentrated 3-isothiazolone solutions were prepared with the compositions and contents given in the following Table 1. As the 3-isothiazolone compound, a mixture of 5-chloro-2-methyl-4-isothiazolin-3-one (CMI) and 2-methyl-4-isothiazolin-3-one (MI) with the mixing ratio of approximately 3:1 was used.

As described in Table 1, the samples of Examples 1 to 13 and Comparative Example 1 were prepared by mixing the 3-isothiazolone compound, sodium nitrate, sodium carbonate, magnesium nitrate (Comparative Example), magnesium oxide (Comparative Example), and water and adding KlO₃, NaBrO₃ and NaClO₂ for preventing precipitation.

TABLE 1
(wt %)

| | Isothiazolone | | | | | | | |
|---|---|---|---|---|---|---|---|---|
| Sample | compound | Mg(NO3)₂ | MgCl₂ | NaNO3 | NaCl | KlO3 | NaBrO3 | NaClO2 |
| Example 1 | 13.84 | — | — | 17.0 | 5.7 | 0.002 | | |
| Example 2 | 13.84 | — | — | 17.0 | 5.7 | 0.005 | | |
| Example 3 | 13.84 | — | — | 17.0 | 5.7 | 0.01 | | |
| Example 4 | 13.84 | — | — | 17.0 | 5.7 | | 0.002 | |
| Example 5 | 13.84 | — | — | 17.0 | 5.7 | | 0.05 | |
| Example 6 | 13.84 | — | — | 17.0 | 5.7 | | 0.20 | |
| Example 7 | 13.84 | — | — | 17.0 | 5.7 | | | 0.01 |
| Example 8 | 13.84 | — | — | 17.0 | 5.7 | | | 0.01 |
| Example 9 | 13.84 | — | — | 17.0 | 5.7 | | | 0.20 |
| Example 10 | 13.84 | — | — | 17.0 | 5.7 | 0.005 | 0.05 | |
| Example 11 | 13.84 | — | — | 17.0 | 5.7 | 0.005 | | 0.1 |
| Example 12 | 13.84 | — | — | 17.0 | 5.7 | | 0.05 | 0.1 |
| Example 13 | 13.84 | — | — | 17.0 | 5.7 | 0.005 | 0.05 | 0.1 |
| Comparative | 13.84 | 18.02 | 4.9 | — | — | — | — | |
| Example 1 | | | | | | | | |

In the above samples of Examples 1 to 13 and Comparative Example 1, the alkali metal carbonate (sodium chloride, potassium chloride) and magnesium chloride are those produced in the composition by sodium carbonate, potassium carbonate, magnesium nitrate and magnesium oxide introduced for pH neutralization.

Specifically, the alkali metal carbonate (sodium carbonate, potassium carbonate, sodium bicarbonate, potassium bicarbonate) introduced as a pH-neutralizing agent exists in the form of an alkali metal chloride salt (NaCl, KCl) in the composition of the present invention.

[Experiment 1]

Salt Shock Test

For samples prepared according to Examples 1 to 13 and Comparative Example 1, whether or not salt shock is caused was examined and the results are described in the following Table 2.

Generally, an isothiazolone composition causing salt shock includes many magnesium ions as a stabilizer. It was confirmed whether or not samples prepared according to Examples 1 to 13 and Comparative Example 1 can be used as a sterilizer for a dispersion or emulsion such as paint acrylate or latex, etc. that sensitively reacts with divalent ions and coagulates, that is, whether or not the composition can stabilize a polymer aqueous dispersion.

For testing, 5 ml of a detecting agent was added to 10 ml of isothiazolone solutions to be tested by droplets. And, as a salt shock detecting agent, a 5% sodium methasilicate aqueous solution (Na₂SiO₃.9H₂O) was used.

TABLE 2

| Sample | Formation of precipitation by salt shock |
|---|---|
| Example 1 | No precipitation after 5 ml added |
| Example 2 | No precipitation after 5 ml added |
| Example 3 | No precipitation after 5 ml added |
| Example 4 | No precipitation after 5 ml added |
| Example 5 | No precipitation after 5 ml added |
| Example 6 | No precipitation after 5 ml added |
| Example 7 | No precipitation after 5 ml added |
| Example 8 | No precipitation after 5 ml added |
| Example 9 | No precipitation after 5 ml added |
| Example 10 | No precipitation after 5 ml added |
| Example 11 | No precipitation after 5 ml added |
| Example 12 | No precipitation after 5 ml added |
| Example 13 | No precipitation after 5 ml added |
| Comparative Example 1 | Precipitation begins to form after 1 ml added |

As can be seen from the Table 2, the salt shock composition forms precipitate, and the non-salt shock types did not form precipitate.

When adding 5 ml of the detecting agent to 10 ml of the sample of Comparative Example 1, a white or gray precipitate formed. However, adding a detecting agent to the sample of Example 1 did not cause formation of a precipitate.

Accordingly, it is apparent that the sample of Comparative Example 1 is of a salt shock type, and that of Example 1 is of a non-salt shock type.

In the above Experiment 1, a salt free isothiazolone solution stabilized by a glycol solvent was used as a control.

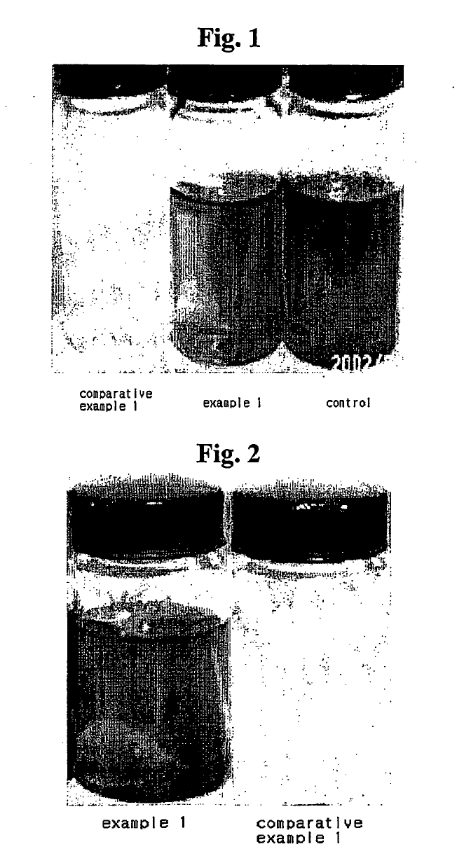

FIG. 1 is a photograph showing the salt shock results of isothiazolone composition samples of Example 1, Comparative Example 1, and a control, in Experiment 1.

In addition, FIG. 2 is a photograph showing the salt shock results of 10-fold dilutions of isothiazolone composition samples of Example 1, and Comparative Example 1 in Experiment 1.

As shown in FIG. 1, the sample of Comparative Example 1 formed a white or gray precipitate, and that of Example 1 did not form a precipitate. And, as shown in FIG. 2, the experiment with the 10-fold dilutions of samples of Example 1 and Comparative Example 1 showed the same results.

Accordingly, it was confirmed that the isothiazolone composition that does not include an alkali earth metal salt of a divalent metal such as magnesium chloride and instead is an isothiazolone composition stabilized by an alkali metal nitrate salt, can stabilize an aqueous high concentration polymer dispersion.

[Experiment 2]

Salt Shock Generation Test in Paint Acrylate Emulsion

Whether or not the samples of Examples 1 to 13 and Comparative Example 1 caused salt shock was examined by the same method as the Experiment 1, except that, in order for practical confirmation, after adding the samples of Examples 1 to 13 and Comparative Example 1 to a paint acrylate emulsion in a ratio of 5 ml:10 ml, whether or not coagulation (precipitation) occurred as a result of salt shock was examined and the results are described in the following Table 3.

TABLE 3

| Sample | Formation of coagulation (precipitation) by salt shock |
|---|---|
| Example 1 | No coagulation (precipitation) after 5 ml added |
| Example 2 | No coagulation (precipitation) after 5 ml added |
| Example 3 | No coagulation (precipitation) after 5 ml added |
| Example 4 | No coagulation (precipitation) after 5 ml added |
| Example 5 | No coagulation (precipitation) after 5 ml added |
| Example 6 | No coagulation (precipitation)n after 5 ml added |
| Example 7 | No coagulation (precipitation) after 5 ml added |
| Example 8 | No coagulation (precipitation) after 5 ml added |
| Example 9 | No coagulation (precipitation) after 5 ml added |
| Example 10 | No coagulation (precipitation) after 5 ml added |
| Example 11 | No coagulation (precipitation) after 5 ml added |
| Example 12 | No coagulation (precipitation) after 5 ml added |
| Example 13 | No coagulation (precipitation) after 5 ml added |
| Comparative | Coagulation (precipitation) begins to form after 1 ml added |
| Example 1 | |

As can be seen from the Table 3, the salt shock type composition coagulated (precipitation), but the non-salt shock type compositions did not coagulate (precipitation).

The sample of Comparative Example 1 coagulated (precipitation) by salt shock when introduced in a polymer aqueous dispersion paint acrylate emulsion.

Accordingly, it can be confirmed that the sample of Comparative Example 1 is of a salt shock type, and those of Examples 1 to 13 are of non-salt shock types.

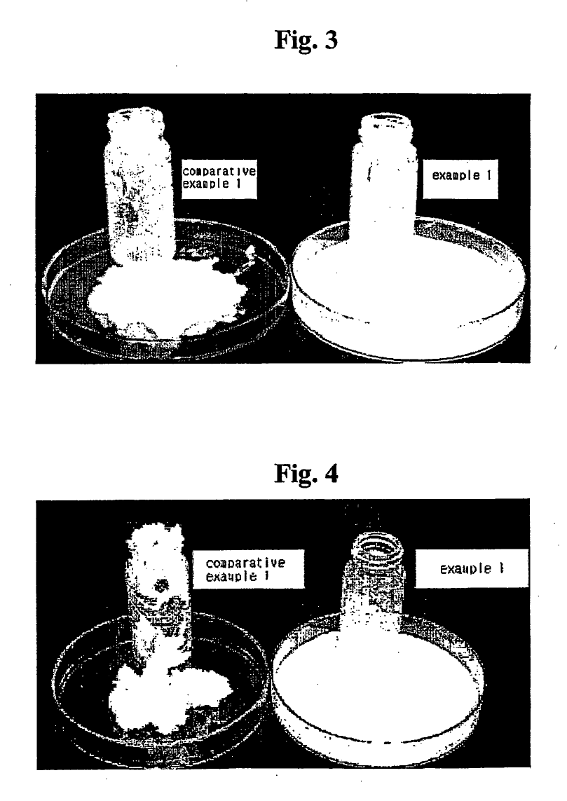

FIG. 3 is a photograph showing the salt shock results of isothiazolone composition samples of Example 1 and Comparative Example 1.

As shown in FIG. 3, the paint acrylate emulsion into which the isothiazolone composition of Comparative Example 1 was introduced was seriously coagulated, and the paint acrylate emulsion into which the isothiazolone composition of Example 1 was introduced maintained a solution state without coagulation.

Accordingly, it can be identified that an isothiazolone composition that does not include an alkali earth metal salt of a divalent metal such as magnesium chloride, that is, an isothiazolone composition stabilized by an alkali metal nitrate salt can stabilize a polymer aqueous dispersion of a high concentration, and thus it can be used for a process such as let down during preparation of paint, to which the existing commercial products cannot be applied.

[Experiment 3]

Salt Shock Test in Latex Emulsion

Whether or not the samples of Examples 1 to 13 and Comparative Example 1 caused salt shock was examined by the same method in Experiment 1, except that, for practical confirmation, after adding the samples of Examples 1 to 13 and Comparative Example 1 to a latex emulsion in a ratio of 5 ml:10 ml, whether or not coagulation (precipitation) by salt shock occurred was examined and the results are presented in the following Table 4.

TABLE 4

| Sample | Formation of coagulation (precipitation) by salt shock |
|---|---|
| Example 1 | No coagulation (precipitation) after 5 ml added |
| Example 2 | No coagulation (precipitation) after 5 ml added |
| Example 3 | No coagulation (precipitation) after 5 ml added |
| Example 4 | No coagulation (precipitation) after 5 ml added |
| Example 5 | No coagulation (precipitation) after 5 ml added |
| Example 6 | No coagulation (precipitation)n after 5 ml added |
| Example 7 | No coagulation (precipitation) after 5 ml added |
| Example 8 | No coagulation (precipitation) after 5 ml added |
| Example 9 | No coagulation (precipitation) after 5 ml added |
| Example 10 | No coagulation (precipitation) after 5 ml added |
| Example 11 | No coagulation (precipitation) after 5 ml added |
| Example 12 | No coagulation (precipitation) after 5 ml added |
| Example 13 | No coagulation (precipitation) after 5 ml added |
| Comparative | Coagulation (precipitation) begins to form after 1 ml added |
| Example 1 | |

As can be seen from the Table 4, the salt shock type composition formed coagulation (precipitation), but the non-salt shock type compositions did not form coagulation (no precipitation).

The sample of Comparative Example 1 formed coagulation (precipitation) by salt shock, when introduced in a polymer aqueous dispersion of latex emulsion. However, those of Examples 1 to 13 did not form coagulation (no precipitation).

Accordingly, it can be identified that the sample of Comparative Example 1 is of a salt shock type, but those of Examples 1 to 13 are of non-salt shock types.

FIG. 4 is a photograph showing the salt shock results of isothiazolone composition samples of Example 1 and Comparative Example 1 in Experiment 3.

As shown in FIG. 4, the latex emulsion into which the isothiazolone composition of Comparative Example 1 was introduced was seriously coagulated, and the latex emulsion into which the sample of Example 1 was introduced maintained a solution state without coagulation.

[Experiment 4]

Precipitation Formation Test

For the samples prepared according to Examples 1 to 13 and Comparative Example 1, a storage stability test was conducted and the results are described in the following Table 5.

In order to confirm storage stability, the isothiazolone compositions (samples of Examples 1 to 13 and Comparative Example 1) were kept in an oven at 65° C. for 28 days, and the concentrations of main ingredients (MI, CMI) remaining in the compositions were analyzed using HLPC. The results are described in the following Table 5.

TABLE 5

| | | Changes in | | | | |
|---|---|---|---|---|---|---|
| | | controls of valid ingredients at 65° C. | | | | |
| | | | | Total | | |
| | Days of | | | (CMI + | Ratio | |
| Sample | storage | MI | CMI | MI) | (CMI/MI) | % |
| Example 1 | Initial Stage | 3.43 | 10.41 | 13.84 | 3.04 | 100 |
| | 28^{th} days | 3.12 | 7.87 | 10.99 | 2.52 | 83.0 |
| Example 2 | Initial Stage | 3.43 | 10.41 | 13.84 | 3.04 | 100 |
| | 28^{th} days | 3.15 | 8.11 | 11.26 | 2.57 | 84.7 |
| Example 3 | Initial Stage | 3.43 | 10.41 | 13.84 | 3.04 | 100 |
| | 28^{th} days | 3.11 | 8.20 | 11.31 | 2.64 | 86.8 |
| Example 4 | Initial Stage | 3.43 | 10.41 | 13.84 | 3.04 | 100 |
| | 28^{th} days | 3.09 | 7.63 | 10.72 | 2.47 | 81.3 |
| Example 5 | Initial Stage | 3.43 | 10.41 | 13.84 | 3.04 | 100 |
| | 28^{th} days | 3.17 | 8.31 | 11.48 | 2.62 | 86.3 |
| Example 6 | Initial Stage | 3.43 | 10.41 | 13.84 | 3.04 | 100 |
| | 28^{th} days | 3.15 | 8.11 | 11.34 | 2.62 | 85.6 |
| Example 7 | Initial Stage | 3.43 | 10.41 | 13.84 | 3.04 | 100 |
| | 28^{th} days | 3.10 | 7.70 | 10.80 | 2.48 | 81.8 |
| Example 8 | Initial Stage | 3.43 | 10.41 | 13.84 | 3.04 | 100 |
| | 28^{th} days | 3.10 | 7.72 | 10.82 | 2.49 | 82 |
| Example 9 | Initial Stage | 3.43 | 10.41 | 13.84 | 3.04 | 100 |
| | 28^{th} days | 3.11 | 7.78 | 10.90 | 2.50 | 82.4 |
| Example 10 | Initial Stage | 3.43 | 10.41 | 13.84 | 3.04 | 100 |
| | 28^{th} days | 3.32 | 10.07 | 13.39 | 3.03 | 100 |
| Example 11 | Initial Stage | 3.43 | 10.41 | 13.84 | 3.04 | 100 |
| | 28^{th} days | 3.34 | 10.15 | 13.49 | 3.04 | 100 |
| Example 12 | Initial Stage | 3.43 | 10.41 | 13.84 | 3.04 | 100 |
| | 28^{th} days | 3.03 | 9.22 | 12.25 | 3.04 | 100 |
| Example 13 | Initial Stage | 3.43 | 10.41 | 13.84 | 3.04 | 100 |
| | 28^{th} days | 3.28 | 9.93 | 13.21 | 3.03 | 100 |
| Comparative | Initial Stage | 3.46 | 10.44 | 13.90 | 3.01 | 100 |
| Example 1 | 28^{th} days | 3.10 | 7.30 | 10.39 | 2.36 | 78 |

As can be seen from the Table 5, the sample of Comparative Example 1 showed a large change in concentration of main ingredients after being stored at 65° C. for 28 days, thus the ratio of CMI/MI decreased and precipitate formed. Since, of the two main ingredients of the isothiazolone solution, i.e. CMI and MI, the stability of CMI is lower than that of MI, the result that the initial ratio of 3:1 decreased means that CMI precipitated by the amount of decrease.

Meanwhile, the samples of Examples 1 to 13, which were prepared by adding KlO₃, NaBrO₃, and NaClO₂ as stabilizers, showed minor changes in concentrations of main ingredients after being stored for 28 days, and particularly when the mixture of KlO₃, NaBrO₃ and NaClO₂ of appropriate ratios were added, there was little change of concentrations of main ingredients, and the ratio of CMI/MI was large, hence a stabilized isothiazolone composition in which precipitation does not occur could be obtained.

Accordingly, from the above experiment, it can be confirmed that a more stabilized 3-isothiazolone composition can be obtained by introducing a small amount of iodate, metal bromate, and chlorite in combination of appropriate ratios, compared to when using each of the compounds alone.

The isothiazolone composition of the present invention can be stably used as a sterilizer in a dispersion or emulsion such as paint acrylate or latex, etc. because it does not include an alkali earth metal salt of a divalent metal such as magnesium chloride, which causes salt shock, and thus it can effectively inhibit growth of harmful microorganisms, and it has superior storage stability due to its low precipitation forming rate.

1. An isothiazolone composition comprising:
(a) a 3-isothiazolone compound represented by 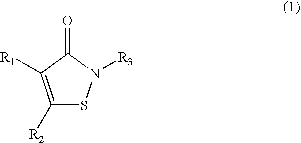
where,
R₁ and R₂ are each independently a hydrogen atom, a halogen atom, a C₁-C₄ alkyl group or an aryl group;
R₃ is a hydrogen atom, a C₁-C₁₈ alkyl group, a c₂-C₁₈ alkenyl group, a c₂-C₁₈ alkynyl group, a C₃-C₁₂ cycloalkyl group having trigonal to octagonal rings, a C₁₀-C₂₄ aralkyl group, or a c₁-C₂₄ aryl group;
(b) an alkali metal nitrate;
(c) an alkali metal chloride; and
(d) a solvent.

2. The isothiazolone composition according to claim 1, wherein the (a) 3-isothiazolone compound represented by the Chemical Formula 1 is selected from the group consisting of 5-chloro-2-methyl-4-isothiazolin-3-one, 2-methyl-4-isothiazolin-3-one, 4,5-dichloro-2-methyl-4-isothiazolin-3-one, 5-chloro-2-n-octyl-3-isothiazolone, 4,5-dichloro-2-n-octyl-3-isothiazolone and benzisothiazolone.

3. The isothiazolone composition according to claim 1, wherein the (b) alkali metal nitrate is selected from the group consisting of lithium nitrate, sodium nitrate and potassium nitrate.

4. The isothiazolone composition according to claim 1, wherein the (c) alkali metal chloride is selected from a group consisting of sodium chloride and potassium chloride.

5. The isothiazolone composition according to claim 1, wherein the (d) solvent is selected from a group consisting of water, ethylene glycol, propyleneglycol, dipropyleneglycol, polypropyleneglycol, diethyleneglycol, triethyleneglycol, 1,5-pentanediol, 2,4-pentanediol and benzyl alcohol.

6. The isothiazolone composition according to claim 1, comprising:
(a) 0.1 to 20 wt % of the 3-isothiazolone compound;
(b) 0.1 to 25 wt % of the alkali metal nitrate;
(c) 0.001 to 20 wt % of the alkali metal chloride; and
(d) the solvent making up the balance.

7. The isothiazolone composition according to claim 6, wherein the (c) alkali metal chloride is present in an amount of 0.1 to 15 wt %, based on the weight of the isothiazolone composition.

8. The isothiazolone composition according to claim 6, wherein the (c) alkali metal chloride is present in an amount of 0.1 to 10 wt %, based on the weight of the isothiazolone composition.

9. The isothiazolone composition according to claim 1, which further comprises a precipitation-preventing agent selected from a group consisting of (e) iodic acid, periodic acid, iodate, periodate or a mixture thereof; (f) metal bromate; and (g) chlorite.

10. The isothiazolone composition according to claim 9, wherein the (e) iodic acid, periodic acid, iodate, periodate, or a mixture thereof is present in an amount of 0.0001 to 0.01 wt %, based on the weight of the isothiazolone composition.

11. The isothiazolone composition according to claim 9, wherein the (e) iodic acid, periodic acid, iodate, periodate or a mixture thereof is present in an amount of 0.001 to 0.005 wt %, based on the weight of the isothiazolone composition.

12. The isothiazolone composition according to claim 9, wherein the (f) metal bromate is present in an amount of 0.001 to 1.0 wt %, based on the weight of the isothiazolone composition.

13. The isothiazolone composition according to claim 9, wherein the (f) metal bromate is present in an amount of 0.001 to 0.2 wt %, based on the weight of the isothiazolone composition.

14. The isothiazolone composition according to claim 9, wherein the (g) chlorite is present in an amount of 0.001 to 10 wt %, based on the weight of the isothiazolone composition.

15. The isothiazolone composition according to claim 9, wherein the (g) chlorite is present in an amount of 0.01 to 1 wt %, based on the weight of the isothiazolone composition.

16. The isothiazolone composition according to claim 9, wherein the (g) chlorite is present in an amount of 0.05 to 0.2 wt %, based on the weight of the isothiazolone composition.

17. The isothiazolone composition according to claim 9, wherein the (e) iodic acid, periodic acid, iodate, periodate or a mixture thereof is selected from a group consisting of lithium iodate, sodium iodate, potassium iodate, ammonium iodate, lithium periodate, sodium periodate, potassium periodate, ammonium periodate, iodic acid, periodic acid dihydrate and a mixture thereof.

18. The isothiazolone composition according to claim 9, wherein the (f) metal bromate is sodium bromate or potassium bromate.

19. The isothiazolone composition according to claim 9, wherein the (g) chlorite is sodium chlorite or potassium chlorite.

20. A method for stabilizing isothiazolone comprising adding (a) 0.1 to 20 wt % of a 3-isothiazolone compound represented by 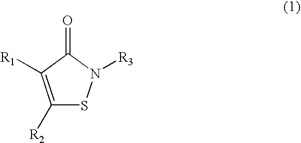
where,
R₁ and R₂ are each independently a hydrogen atom, a halogen atom, a C₁-C₄ alkyl group or an aryl group;
R₃ is a hydrogen atom, a C₁-C₁₈ alkyl group, a C₂-C₁₈ alkenyl group, a C₂-C₁₈ alkynyl group, a C₃-C₁₂ cycloalkyl group having trigonal to octagonal rings, a C₁₀-C₂₄ aralkyl group, or a C₁₀-C₂₄ aryl groups;
(b) 0.1 to 25 wt % of an alkali metal nitrate; and (c) 0.001 to 20 wt % of an alkali metal carbonate, to (d) a solvent making up the balance, to prepare a mixed solution.

21. The method for stabilizing isothiazolone according to claim 20, wherein the (a) 3-isothiazolone compound of the Chemical Formula 1 is selected from a group consisting of 5-chloro-2-methyl-4-isothiazolin-3-one, 2-methyl-4-isothiazolin-3-one, 4,5-dichloro-2-methyl-4-isothiazolin-3-one, 5-chloro-2-n-octyl-3-isothiazolone, 4,5-dichloro-2-n-octyl-3-isothiazolone and benzisothiazolone.

22. The method for stabilizing isothiazolone according to claim 20, wherein the (b) alkali metal nitrate is selected from a group consisting of lithium nitrate, sodium nitrate and potassium nitrate.

23. The method for stabilizing isothiazolone according to claim 20, wherein the (c) alkali metal carbonate is selected from a group consisting of sodium carbonate, potassium carbonate, sodium bicarbonate and potassium bicarbonate.

24. The method for stabilizing isothiazolone according to claim 20, wherein the (d) solvent is selected from a group consisting of water, ethyleneglycol, propyleneglycol, dipropyleneglycol, polypropyleneglycol, diethyleneglycol, triethyleneglycol, 1,5-pentanediol, 2,4-pentanediol and benzyl alcohol.

25. The method for stabilizing isothiazolone according to claim 20, wherein the (c) alkali metal carbonate is present in an amount of 0.01 to 15 wt %, based on the weight of the isothiazolone composition.

26. The method of stabilizing isothiazolone according to claim 20, wherein the (c) alkali metal carbonate is present in an amount of 0.1 to 10 wt %, based on the weight of the isothiazolone composition.

27. The method for stabilizing isothiazolone according to claim 20, which further comprises adding a precipitation-preventing agent selected from a group consisting of (e) iodic acid, periodic acid, iodate, periodate and a mixture thereof; (f) metal bromate; and (g) chlorite, to the mixed solution.

28. The method for stabilizing isothiazolone according to claim 27, wherein the (e) iodic acid, periodic acid, iodate, periodate or a mixture thereof is contained in an amount of 0.0001 to 0.01 wt %, based on the weight of the isothiazolone composition.

29. The method for stabilizing isothiazolone according to claim 27, wherein the (e) iodic acid, periodic acid, iodate, periodate, or a mixture thereof is present in an amount of 0.001 to 0.005 wt %, based on the weight of the isothiazolone composition.

30. The method for stabilizing isothiazolone according to claim 27, wherein the (f) metal bromate is present in an amount of 0.001 to 1.0 wt %, based on the weight of the isothiazolone composition.

31. The method for stabilizing isothiazolone according to claim 27, wherein the (f) metal bromate is present in an amount of 0.001 to 0.2 wt %, based on the weight of the isothiazolone composition.

32. The method for stabilizing isothiazolone according to claim 27, wherein the (g) chlorite is present in an amount of 0.001 to 10 wt %, based on the weight of the isothiazolone composition.

33. The method for stabilizing isothiazolone according to claim 27, wherein the (g) chlorite is present in an amount of 0.01 to 1 wt %, based on the weight of the isothiazolone composition.

34. The method for stabilizing isothiazolone according to claim 27, wherein the (g) chlorite is present in an amount of 0.05 to 0.2 wt %, based on the weight of the isothiazolone composition.

35. The method for stabilizing isothiazolone according to claim 27, wherein the (e) iodic acid, periodic acid, iodate, periodate or a mixture thereof is selected from a group consisting of lithium iodate, sodium iodate, potassium iodate, ammonium iodate, lithium periodate, sodium periodate, potassium periodate, ammonium periodate, iodic acid, periodic acid dihydrate or a mixture thereof.

36. The method for stabilizing isothiazolone according to claim 27, wherein the (f) metal bromate is sodium bromate or potassium bromate.

37. The method for stabilizing isothiazolone according to claim 27, wherein the (g) chlorite is sodium chlorite or potassium chlorite.
